(19) United States
(12) Patent Application Publication
(10) Pub. No.: US 2026/0248545 A1
(43) Pub. Date: Aug. 27, 2026

(54) RETAINING AND ROUTING ELECTRICAL WIRES IN SURGICAL TOOLS

(71) Applicant: CILAG GMBH INTERNATIONAL, Zug (CH)

(72) Inventors: Austin Michael FISCHER, Cincinnati, OH (US); Christopher William BIRRI, West Chester, OH (US)

(73) Assignee: CILAG GMBH INTERNATIONAL, Zug (CH)

(21) Appl. No.: 19/064,166

(22) Filed: Feb. 26, 2025

(51) Int. Cl. A61B 18/00; A61B 90/00

(52) U.S. Cl. CPC A61B 18/00; A61B 90/08; A61B 2018/00172

(57) ABSTRACT

A surgical tool includes a drive housing with an electrical assembly housed therein, an elongate shaft extending from the drive housing, an end effector arranged at a distal end of the elongate shaft, one or more wires extending from the end effector to the drive housing and terminating at the electrical assembly, and a wire routing system provided within the drive housing. The wire routing system includes an axial retainment feature including one or more overhanging bosses that define a tortuous pathway for the one or more wires to traverse, and a wire channel that guides the one or more wires toward the electrical assembly.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

The motion of the drive cables and corresponding mechanisms inside of the tool driver, however, may present hazardous conditions for any electrical wiring present within the drive housing and extending to the end effector. Further, articulation of the end effector to desired angular positions and configurations may necessitate a minimum level of slack for said electrical wiring to account for the changing path length that must be traversed. The presence of wiring slack within the tool driver and drive housing may exacerbate the dangers to the electrical wiring from the moving components of the tool driver.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIG. 4 is an enlarged isometric view of the distal end of the surgical tool of FIG. 2.

FIG. 5 is a bottom view of the drive housing of FIG. 2, according to one or more embodiments.

FIG. 6 is an exposed isometric view of the interior of the drive housing of FIG. 2, according to one or more embodiments.

FIG. 7 is another exposed isometric view of the interior of the drive housing, according to one or more additional embodiments.

FIG. 8 is an isometric view of an example routing anchor, according to one or more embodiments of the present disclosure.

FIG. 9 is another exposed isometric view of the interior of the drive housing, according to one or more additional embodiments.

FIG. 10A is an enlarged side view of a portion of the wire guide, according to one or more embodiments.

FIG. 10B is an enlarged top view of the wire guide depicting the overhanging bosses with the wires constrained therein, according to one or more embodiments.

FIG. 11 is a partial isometric view of an example electrical connector installed within the drive housing, according to one or more embodiments.

FIG. 12 is a top view of the interior of another example drive housing, according to one or more additional embodiments.

FIG. 13 is a partial isometric view of another example drive housing, in accordance with one or more additional embodiments.

FIG. 14A is an isometric view of another example drive housing, in accordance with one or more additional embodiments.

FIG. 14B is an enlarged end view of a guide component, according to one or more embodiments.

FIG. 14C is an enlarged cross-sectional view of a portion of the drive housing, according to one or more embodiments.

### DETAILED DESCRIPTION

The present disclosure is related to robotic surgical systems and, more particularly, to preventing derailment and binding issues with drive cables of a cable driven surgical tool when slack accumulates in the drive cables.

The present disclosure describes a surgical tool that includes a drive housing including an electrical assembly housed therein, an elongate shaft extending from the drive housing, an end effector arranged at a distal end of the elongate shaft, one or more wires extending from the end effector to the drive housing and terminating at the electrical assembly, and a wire routing system provided within the drive housing. The wire routing system includes an axial retainment feature including one or more overhanging bosses that define a tortuous pathway for the one or more wires to traverse, and a wire channel that guides the one or more wires toward the electrical assembly. The wire routing system maintains a routing pathway for the wires that keeps the wires in predictable positions, thus preventing damage or binding due to catching on movable components within the drive housing.

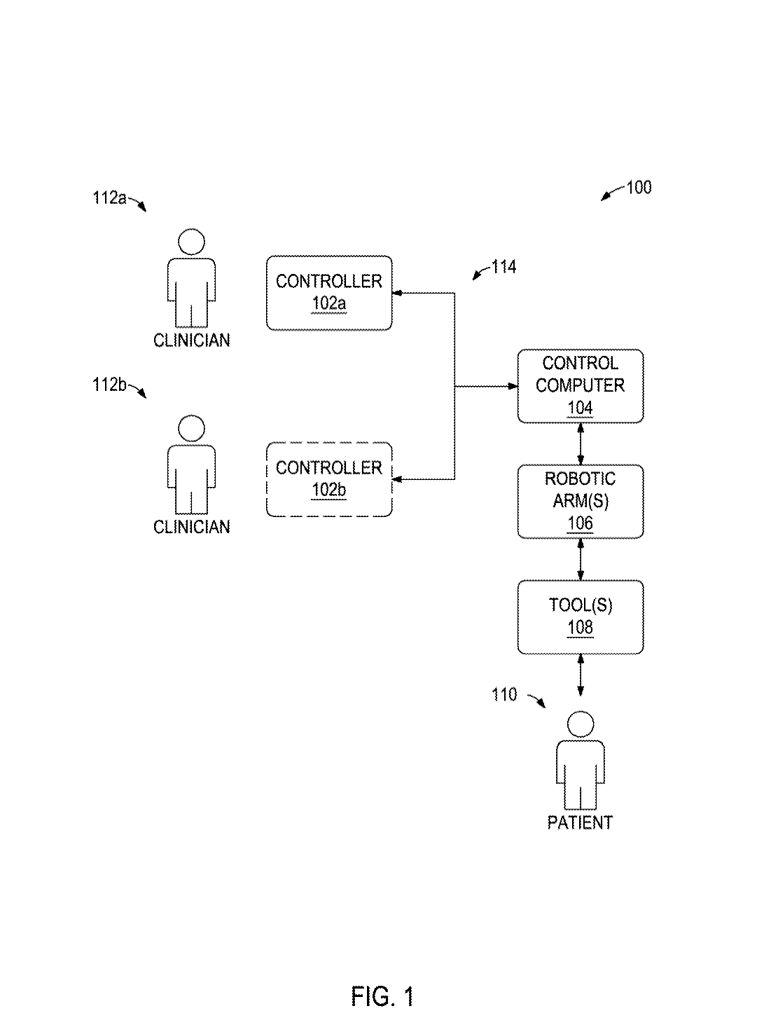

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

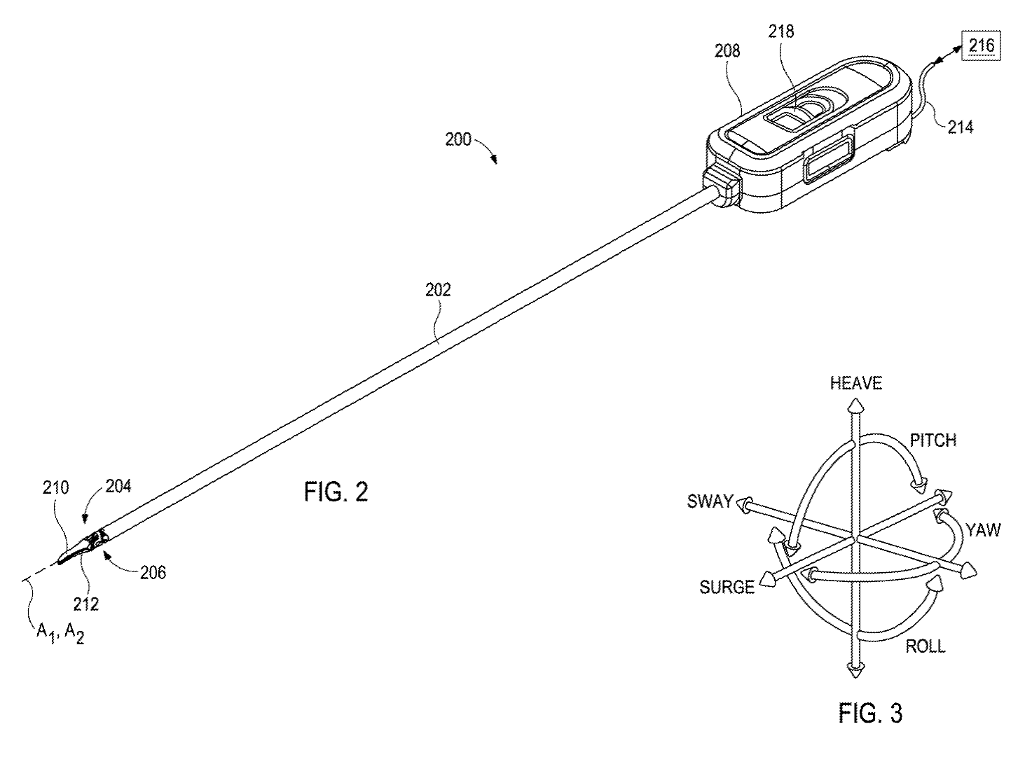

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A₁. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises a combination tissue grasper and vessel sealer that include opposing first (upper) and second (lower) jaws 210, 212 configured to move (articulate) between open and closed positions. As will be appreciated, however, the opposing jaws 210, 212 may alternatively form part of other types of end effectors such as, but not limited to, a surgical scissors, a clip applier, a needle driver, a babcock including a pair of opposed grasping jaws, bipolar jaws (e.g., bipolar Maryland grasper, forceps, a fenestrated grasper, etc.), etc. One or both of the jaws 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator 216 that supplies energy, such as electrical energy (e.g., radio frequency energy), ultrasonic energy, microwave energy, heat energy, or any combination thereof, to the surgical tool 200 and, more particularly, to the end effector 204. Accordingly, the generator 216 may comprise a radio frequency (RF) source, an ultrasonic source, a direct current source, and/or any other suitable type of electrical energy source that may be activated independently or simultaneously.

In applications where the surgical tool 200 is configured for bipolar operation, the power cable 214 will include a supply conductor and a return conductor. Current can be supplied from the generator 216 to an active (or source) electrode located at the end effector 204 via the supply conductor, and current can flow back to the generator 216 via a return electrode located at the end effector 204 via the return conductor. In the case of a bipolar grasper with opposing jaws, for example, the jaws serve as the electrodes where the proximal end of the jaws are isolated from one another and the inner surface of the jaws (i.e., the area of the jaws that grasp tissue) apply the current in a controlled path through the tissue. In applications where the surgical tool 200 is configured for monopolar operation, the generator 216 transmits current through a supply conductor to an active electrode located at the end effector 204, and current is returned (dissipated) through a return electrode (e.g., a grounding pad) separately coupled to a patient's body.

The surgical tool 200 may further include a manual release switch 218 that may be manually actuated by a user (e.g., a surgeon) to override the cable driven system and thereby manually articulate or operate the end effector 204. The release switch 218 is movably positioned on the drive housing 208, and a user is able to manually move (slide) the release switch 218 from a disengaged position, as shown, to an engaged position. In the disengaged position, the surgical tool 200 is able to operate as normal. As the release switch 218 moves to the engaged position, however, various internal component parts of the drive housing 208 are simultaneously moved, thereby resulting in the jaws 210, 212 opening, which might prove beneficial for a variety of reasons. In some applications, for example, the release switch 218 may be moved in the event of an electrical disruption that renders the surgical tool 200 inoperable. In such applications, the user would be able to manually open the jaws 210, 212 and thereby release any grasped tissue and remove the surgical tool 200. In other applications, the release switch 218 may be actuated (enabled) to open the jaws 210, 212 in preparation for cleaning and/or sterilization of the surgical tool 200.

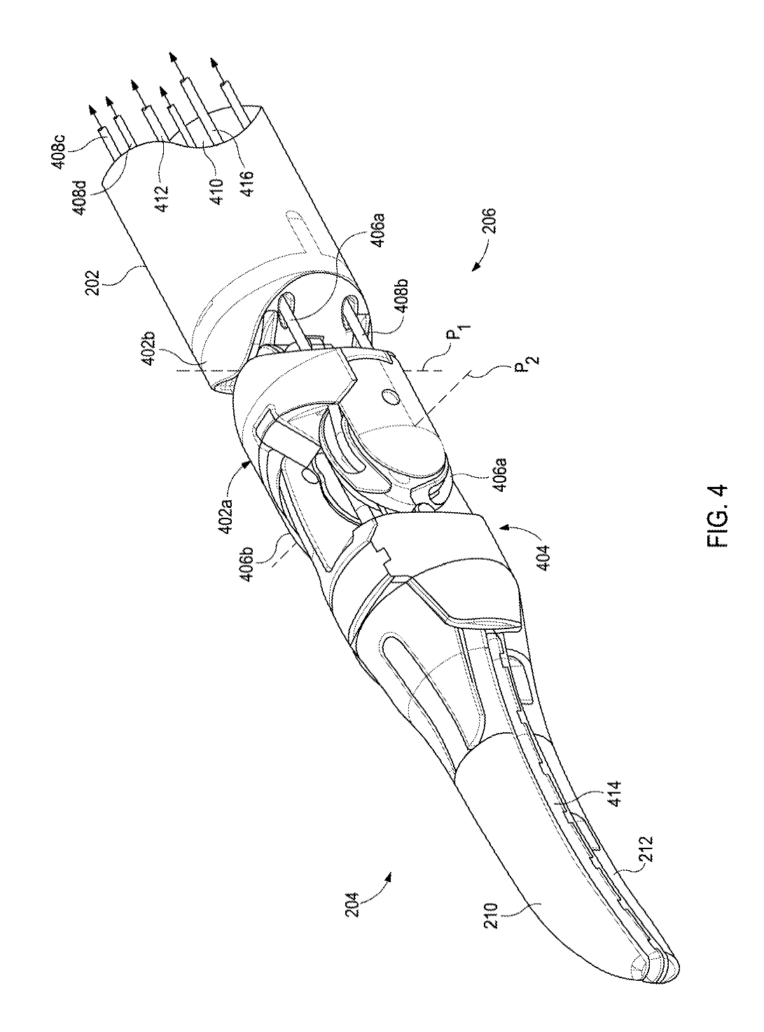

FIG. 4 is an enlarged isometric view of the distal end of the surgical tool 200. More specifically, FIG. 4 depicts an enlarged view of the end effector 204 and the wrist 206, with the jaws 210, 212 of the end effector 204 in the closed position. The wrist 206 operatively couples the end effector 204 to the shaft 202. In some embodiments, however, a shaft adapter may be directly coupled to the wrist 206 and otherwise interpose the shaft 202 and the wrist 206. Accordingly, the wrist 206 may be operatively coupled to the shaft 202 either through a direct coupling engagement where the wrist 206 is directly coupled to the distal end of the shaft 202, or an indirect coupling engagement where a shaft adapter interposes the wrist 206 and the distal end of the shaft 202. As used herein, the term “operatively couple” refers to a direct or indirect coupling engagement between two components.

To operatively couple the end effector 204 to the shaft 202, the wrist 206 includes a first or “distal” clevis 402a and a second or “proximal” clevis 402b. The clevises 402a,b are alternatively referred to as “articulation joints” of the wrist 206 and extend from the shaft 202 (or alternatively a shaft adapter). The clevises 402a,b are operatively coupled to facilitate articulation of the wrist 206 relative to the shaft 202. As illustrated, the wrist 206 also includes a linkage 404 arranged distal to the distal clevis 402a and operatively mounted to the jaws 210, 212.

The proximal end of the distal clevis 402a may be rotatably mounted or pivotably coupled to the proximal clevis 402b at a first pivot axis P₁ of the wrist 206. In some embodiments, an axle may extend through the first pivot axis P₁ and the distal and proximal clevises 402a,b may be rotatably coupled via the axle. In other embodiments, however, such as is depicted in FIG. 4, the distal and proximal clevises 402a,b may be engaged in rolling contact, such as via an intermeshed gear relationship that allows the clevises 402a,b to rotate relative to each other similar to a rolling joint.

First and second pulleys 406a and 406b may be rotatably mounted to the distal end of the distal clevis 402a at a second pivot axis P₂ of the wrist 206. The linkage 404 may be arranged distal to the second pivot axis P₂ and operatively mounted to the jaws 210, 212. The first pivot axis P₁ is substantially perpendicular (orthogonal) to the longitudinal axis A₁ of the shaft 202, and the second pivot axis P₂ is substantially perpendicular (orthogonal) to both the longitudinal axis A₁ and the first pivot axis P₁. Movement of the end effector 204 about the first pivot axis P₁ provides “yaw” articulation of the wrist 206, and movement about the second pivot axis P₂ provides “pitch” articulation of the wrist 206.

A plurality of drive cables, shown as drive cables 408a, 408b, 408c, and 408d, extend longitudinally within a lumen 410 defined by the shaft 202 (or a shaft adaptor) and extend at least partially through the wrist 206. The drive cables 408a-d may form part of the cable driven motion system housed within the drive housing 208 (FIG. 2), and may comprise cables, bands, lines, cords, wires, woven wires, ropes, strings, twisted strings, elongate members, belts, shafts, flexible shafts, drive rods, or any combination thereof. The drive cables 408a-d can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof. While four drive cables 408a-d are depicted in FIG. 4, more or less than four may be employed, without departing from the scope of the disclosure.

The drive cables 408a-d extend proximally from the end effector 204 and the wrist 206 toward the drive housing 208 (FIG. 2) where they are operatively coupled to various actuation mechanisms or devices that facilitate longitudinal movement (translation) of the drive cables 408a-d within the lumen 410. Selective actuation of the drive cables 408a-d applies tension (i.e., pull force) to the given drive cable 408a-d in the proximal direction, which urges the given drive cable 408a-d to translate longitudinally within the lumen 410.

In the illustrated embodiment, the drive cables 408a-d each extend longitudinally through the proximal clevis 402b. The distal end of each drive cable 408a-d terminates at the first or second pulleys 406a,b, thus operatively coupling each drive cable 408a-d to the end effector 204. In some embodiments, the distal ends of the first and second drive cables 408a,b may be coupled to each other and terminate at the first pulley 406a, and the distal ends of the third and fourth drive cables 408c,d may be coupled to each other and terminate at the second pulley 406b. In at least one embodiment, the distal ends of the first and second drive cables 408a,b and the distal ends of the third and fourth drive cables 408c,d may each be coupled together at corresponding ball crimps (not shown) mounted to the first and second pulleys 406a,b, respectively.

In at least one embodiment, the drive cables 408a-d may operate “antagonistically”. More specifically, when the first drive cable 408a is actuated (moved), the second drive cable 408b naturally follows as coupled to the first drive cable 408a, and when the third drive cable 408c is actuated, the fourth drive cable 408d naturally follows as coupled to the third drive cable 408c, and vice versa. Antagonistic operation of the drive cables 408a-d can open or close the jaws 210, 212. More specifically, selective actuation of the drive cables 408a-d in other known configurations or coordination will cause the jaws 210, 212 to open or close. Antagonistic operation of the drive cables 408a-d can further cause the end effector 204 to articulate at the wrist 206. More specifically, selective actuation of the drive cables 408a-d in known configurations or coordination can cause the end effector 204 to articulate about one or both of the pivot axes P₁, P₂, thus facilitating articulation of the end effector 204 in both pitch and yaw directions, either individually or simultaneously. Antagonistic operation of the drive cables 408a-d advantageously reduces the number of cables required to provide full wrist 206 motion, and also helps eliminate slack in the drive cables 408a-d, which results in more precise motion of the end effector 204.

In the illustrated embodiment, the end effector 204 is able to articulate (move) in pitch about the second or “pitch” pivot axis P₂, which is located near the distal end of the wrist 206. Thus, the jaws 210, 212 open and close in the direction of pitch. In other embodiments, however, the wrist 206 may alternatively be configured such that the second pivot axis P₂ facilitates yaw articulation of the jaws 210, 212, without departing from the scope of the disclosure.

In some embodiments, an electrical conductor 412 may also extend longitudinally within the lumen 410, through the wrist 206, and terminate at an electrode 414 to supply electrical energy to the end effector 204. In some embodiments, the electrical conductor 412 may comprise a wire, but may alternatively comprise a rigid or semi-rigid shaft, rod, or strip (ribbon) made of a conductive material. The electrical conductor 412 may be entirely or partially covered with an insulative covering (overmold) made of a non-conductive material. Using the electrical conductor 412 and the electrode 414, the end effector 204 may be configured for monopolar or bipolar RF operation.

In the illustrated embodiment, the end effector 204 comprises a combination tissue grasper and vessel sealer that includes a knife (not visible), alternately referred to as a “cutting element” or “blade.” The knife is aligned with and configured to traverse a guide track (not visible) defined longitudinally in one or both of the upper and lower jaws 210, 212. The knife may be operatively coupled to the distal end of a drive rod 416 that extends longitudinally within the lumen 410 and passes through the wrist 206. Longitudinal movement (translation) of the drive rod 416 correspondingly moves the knife within the guide track(s). Similar to the drive cables 408a-d, the drive rod 416 may form part of the actuation systems housed within the drive housing 208 (FIG. 2). Selective actuation of a corresponding drive input will cause the drive rod 416 to move distally or proximally within the lumen 410, and correspondingly move the knife in the same longitudinal direction.

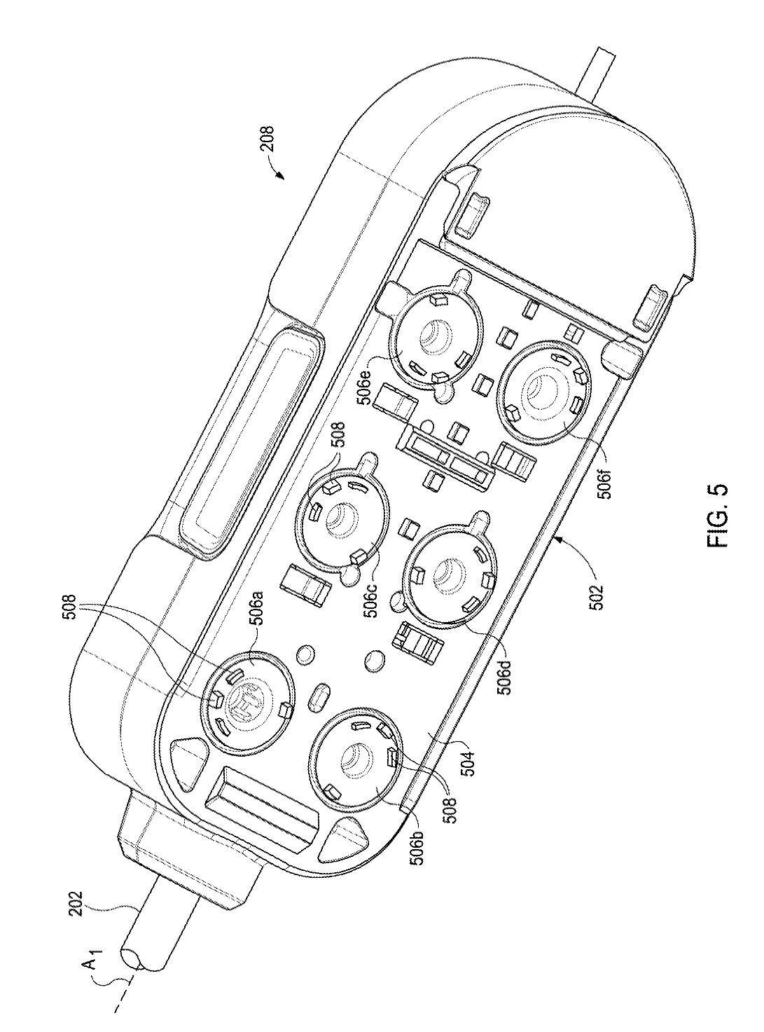

FIG. 5 is a bottom view of the drive housing 208, according to one or more embodiments. As illustrated, the drive housing 208 may include a tool mounting portion 502 used to operatively couple the drive housing 208 to a tool driver of a robotic manipulator. The tool mounting portion 502 may releasably couple the drive housing 208 to a tool driver in a variety of ways, such as by clamping thereto, clipping thereto, or slidably mating therewith. In some embodiments, the tool mounting portion 502 may include an array of electrical connecting pins, which may be coupled to an electrical connection on the mounting surface of the tool driver. While the tool mounting portion 502 is described herein with reference to mechanical, electrical, and magnetic coupling elements, it should be understood that a wide variety of telemetry modalities might be used, including infrared, inductive coupling, or the like.

The tool mounting portion 502 includes and otherwise provides an interface 504 configured to mechanically, magnetically, and/or electrically couple the drive housing 208 to the tool driver. As illustrated, the interface 504 includes and supports a plurality of drive inputs, shown as drive inputs 506a, 506b, 506c, 506d, 506e, and 506f. Each drive input 506a-f comprises a rotatable disc configured to align with and couple to a corresponding actuator or “drive output” of a tool driver, such that rotation (actuation) of a given drive output drives (rotates) a corresponding one of the drive inputs 506a-f. Each drive input 506a-f may provide or define one or more surface features 508 configured to align with mating surface features provided on the corresponding drive output. The surface features 508 can include, for example, various protrusions and/or indentations that facilitate a mating engagement. In some embodiments, some or all of the drive inputs 506a-f may include one surface feature 508 that is positioned closer to an axis of rotation of the associated drive input 506a-f than the other surface feature(s) 508. This may help to ensure positive angular alignment of each drive input 506a-f.

In some embodiments, actuation of the first drive input 506a may be configured to control rotation of the shaft 202 about its longitudinal axis A₁. The shaft 202 may be rotated clockwise or counter-clockwise depending on the rotational actuation of the first drive input 506a. In some embodiments, actuation of the second, third, fourth, and fifth drive inputs 506b-e may be configured to operate movement (axial translation) of the drive cables 408a-d (FIG. 4), which results in the actuation of the wrist 206 (FIG. 4) and/or articulation (operation) of the end effector 204 (FIG. 4). In some embodiments, actuation of the sixth drive input 506f may be configured to advance and retract the drive rod 416 (FIG. 4), and thereby correspondingly advance or retract the knife at the end effector 204. Each of the drive inputs 506a-f may be actuated based on user inputs communicated to the tool driver coupled to the interface 504, and the user inputs may be received via a computer system incorporated into the robotic surgical system.

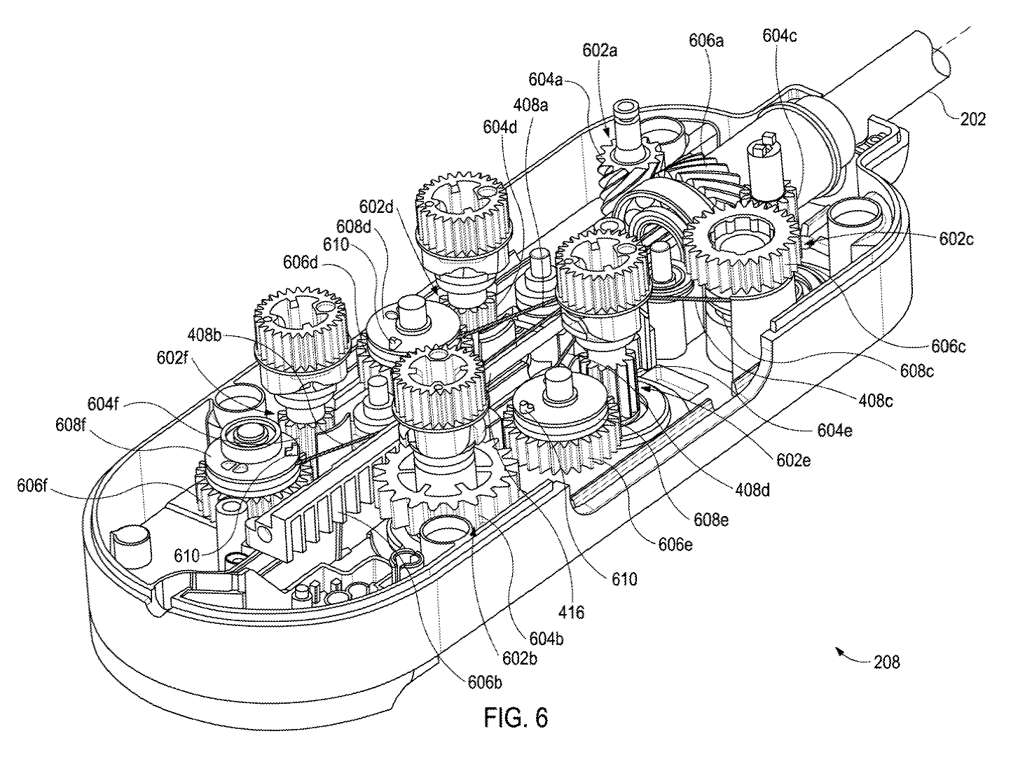

FIG. 6 is an exposed isometric view of the interior of the drive housing 208, according to one or more embodiments. Several component parts that may be otherwise contained within the drive housing 208 are not shown in FIG. 6 to enable discussion of the depicted component parts. As illustrated, the drive housing 208 houses and otherwise contains a plurality of capstan assemblies operable to operate surgical tool 200 (FIG. 2). In particular, a first capstan assembly 602a is contained (housed) within the drive housing 208 and may include a drive gear 604a, which may be operatively coupled to or extend from the first drive input 506a (FIG. 5) such that actuation of the first drive input 506a results in rotation of the drive gear 604a. In the illustrated embodiment, the drive gear 604a comprises a worm gear, which may be configured to mesh and interact with a driven gear 606a secured within the drive housing 208 and operatively coupled to the shaft 202 such that rotation of the driven gear 606a correspondingly rotates the shaft 202. Accordingly, actuation of the first capstan assembly 602a, via actuation of the first drive input 506a, will drive the driven gear 606a and thereby control rotation of the elongated shaft 202 about the longitudinal axis A₁.

The drive housing 208 further contains or houses a second capstan assembly 602b, which may include a drive gear 604b operatively coupled to or extending from the sixth drive input 506f (FIG. 5) such that actuation of the sixth drive input 506f results in rotation of the drive gear 604b. The drive gear 604b is arranged to intermesh with a driven gear 606b positioned within the drive housing 208. In the illustrated embodiment, the driven gear 606b comprises a rack gear longitudinally translatable within the drive housing 208 as acted upon by the drive gear 604b. The drive rod 416 may be operatively coupled to the driven gear 606b and extend distally therefrom to the end effector 204 (FIGS. 2 and 4). Accordingly, actuation of the second capstan assembly 602b, via actuation of the sixth drive input 506f, will cause the driven gear 606b to longitudinally translate and correspondingly advance or retract the drive rod 416 and the knife coupled to the end of the drive rod 416 at the end effector 204.

The drive housing 208 further contains or houses third, fourth, fifth, and sixth capstan assemblies 602c, 602d, 602e, and 602f, alternately be referred to as “drive cable” capstan assemblies since they are operable to actuate the drive cables 408a-d, as described below. While four “drive cable” capstan assemblies 602c-f are depicted in FIG. 6, alternative embodiments may include more or less than four, depending on how many drive cables 408a-d are used.

In the illustrated embodiment, the third capstan assembly 602c is actuated through operation (rotation) of the second drive input 506b (FIG. 5), the fourth capstan assembly 602d is actuated through operation (rotation) of the third drive input 506c (FIG. 5), the fifth capstan assembly 602e is actuated through operation (rotation) of the fourth drive input 506d (FIG. 5), and the sixth capstan assembly 602f is actuated through operation (rotation) of the fifth drive input 506e (FIG. 5). As illustrated, each capstan assembly 602c-f includes a drive gear 604c, 604d, 604e, and 604f that is coupled to or extends from the corresponding drive input 506b-e, respectively, such that actuation (rotation) of the drive input 506b-e correspondingly rotates the associated drive gear 604c-f, respectively.

Moreover, each drive gear 604c-f is positioned to mesh and interact with a corresponding driven gear 606c, 606d, 606e, and 606f rotatably mounted within the drive housing 208. Each driven gear 606c-f includes or is otherwise coupled to a corresponding cable pulley 608c, 608d, 608e, and 608f, and each cable pulley 608c-f is configured to be operatively coupled to (e.g., has wrapped there around, at least partially) a corresponding one of the drive cables 408a-d. In the illustrated embodiment, the first drive cable 408a terminates at cable pulley 608d ultimately driven by actuation of the fourth capstan assembly 602d, the second drive cable 408b terminates at cable pulley 608f ultimately driven by actuation of the sixth capstan assembly 602f, the third drive cable 408c terminates at cable pulley 608c ultimately driven by actuation of the third capstan assembly 602c, and the fourth drive cable 408d terminates at cable pulley 608e ultimately driven by actuation of the fifth capstan assembly 602e.

Accordingly, actuation of the fourth capstan assembly 602d (via operation of the third drive input 506c of FIG. 5) will correspondingly control movement of the first drive cable 408a; actuation of the sixth capstan assembly 602f (via operation of the fifth drive input 506e of FIG. 5) will correspondingly control movement of the second drive cable 408b; actuation of the third capstan assembly 602c (via operation of the second drive input 506b of FIG. 5) will correspondingly control movement of the third drive cable 408c; and actuation of the fifth capstan assembly 602e (via operation of the fourth drive input 506d of FIG. 5) will correspondingly control movement of the fourth drive cable 408d.

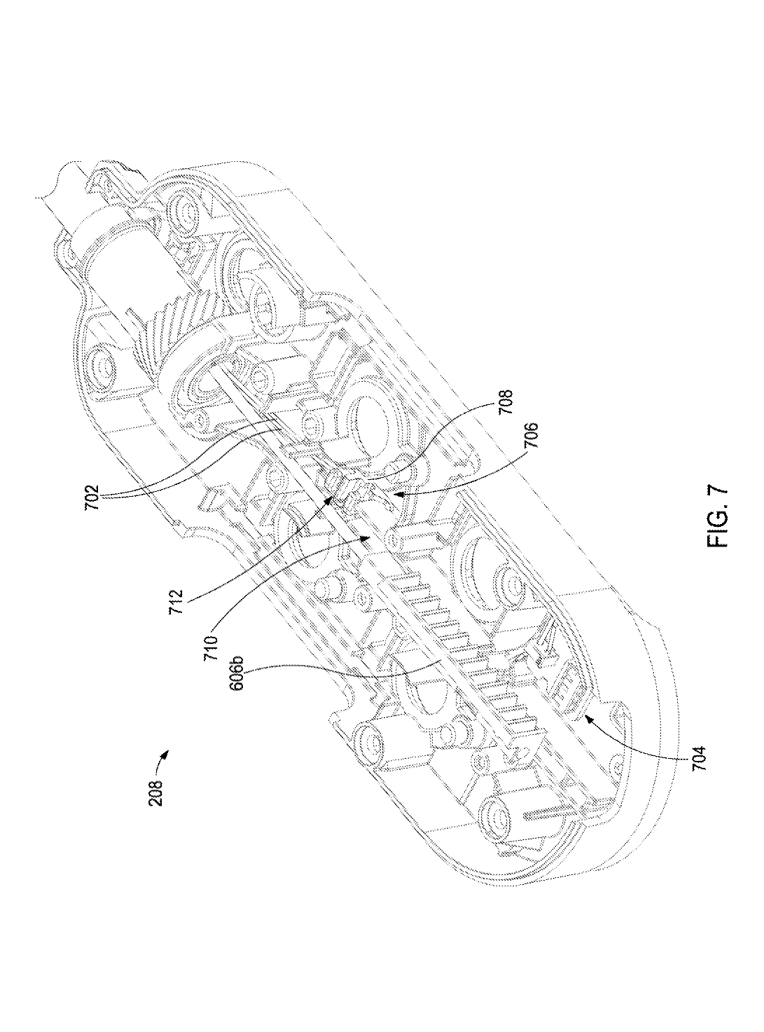

FIG. 7 is another exposed isometric view of the interior of the drive housing 208, according to one or more additional embodiments. Several additional component parts of the drive housing 208 are omitted in FIG. 7 to enable discussion of the remaining component parts. As illustrated, the drive housing 208 may further house one or more wires 702, which extend proximally from the end effector 204 (FIGS. 2 and 4) and are routed through the drive housing 208 to be connected to an electrical assembly 704, also housed within the drive housing 208. The wire(s) 702 may be the same as the electrical conductor 412 (FIG. 4) discussed above, and the electrical assembly 704 may be electrically coupled to the power cable 214 (FIG. 2) to provide power to the wire(s) 702, such that the wire(s) 702 provide an electrical pathway between the power cable 214 and the end effector 204 (FIG. 2) through the drive housing 208. In further embodiments, the wires 702 may enable communication therethrough, such that the wires 702 can provide data transmission alongside the electrical pathway.

In embodiments where the surgical tool 200 (FIG. 2) is configured for bipolar operation, two wires 702 (electrical conductors) will be included. However, in embodiments where the surgical tool 200 is configured for monopolar operation, only one wire 702 (electrical conductor) will be included. In the illustrated embodiment, two wires 702 are included.

During operation of the surgical tool 200 (FIG. 2), rotation and translation of the various component parts may alter the length of the wires 702 extending to the end effector 204 (FIGS. 2 and 4). As the end effector 204 articulates, for example, the total distance (or “path length”) from the drive housing 208 to the end effector 204 will correspondingly increase and decrease. The wires 702 are arranged within the drive housing 208 with a predetermined amount of slack to account for said changes in path length. However, if the wires 702 maintain excess slack within the drive housing 208, various moving component parts included within the drive housing 208, such as the driven gear 606b (or “knife rack”), may contact the slacked portions of the wires 702. Repeated contact against the wires 702 could damage the wires 702, as well as damage the internal mechanisms stored in the drive housing 208. In extreme cases, repeated or continuous contacting of the wires 702 could result in the wires 702 becoming captured or snagged on the moving components, which would effectively render the surgical tool 200 inoperable.

According to embodiments of the present disclosure, the drive housing 208 may further include a wire routing system 706 that provides a pathway for the protection and retainment of the wires 702 within the drive housing 208. In some embodiments, as illustrated, the wire routing system 706 includes a routing anchor 708 that may be at least partially arranged within a slot 710 included within or otherwise defined by the drive housing 208. The slot 710 may provide a track that guides the driven gear 606b as it reciprocates distally and proximally during actuation. As described in more detail below, the routing anchor 708 may provide a wire channel for the wires 702 to extend beneath the driven gear 606b and the track of the slot 710, and may thereby provide a pathway in which the wires 702 are isolated and protected from movement of at least the driven gear 606b. The routing anchor 708 may further provide an axial retainment feature 712, which enables setting of an available slack length extending towards the distal end of the drive housing 208.

While the routing anchor 708 is described herein as comprising a separate component part that can be mounted to the drive housing 208, it is contemplated herein that the routing anchor 708 may be integrally formed with the drive housing 208. In such embodiments, the routing anchor 708 may be formed (e.g., thermoformed or injection molded) along with the remaining portions of the drive housing, thereby forming a single component that protects the wires 702.

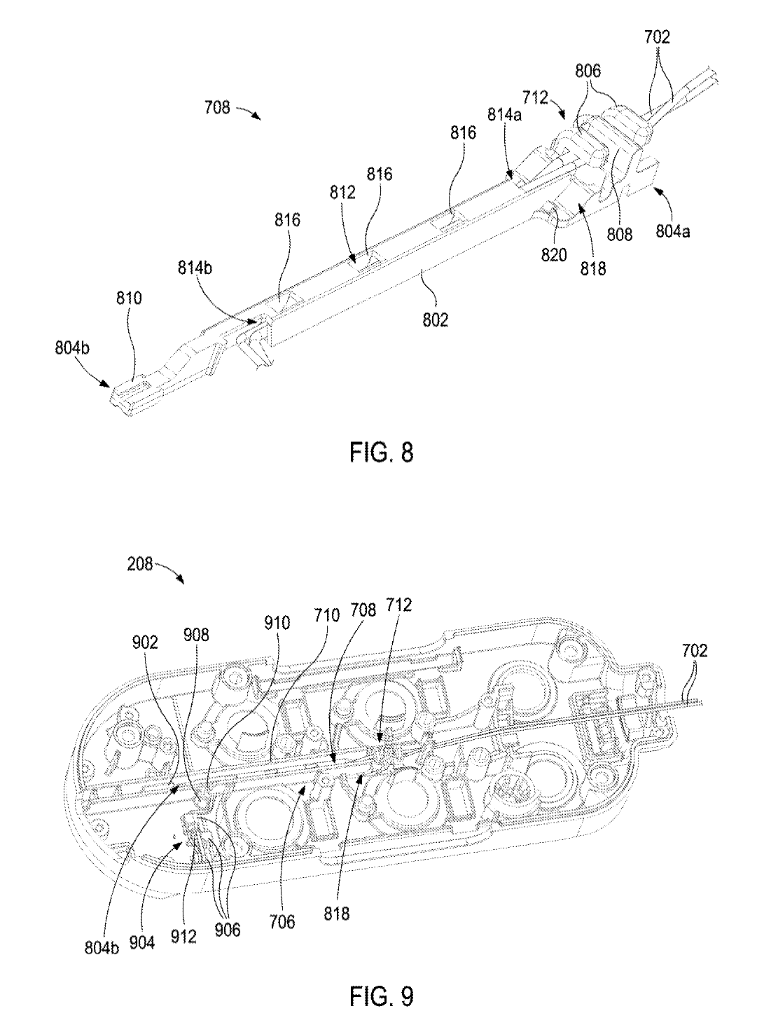

FIG. 8 is an isometric view of an example of the routing anchor 708, according to one or more embodiments of the present disclosure. In the illustrated embodiment, the routing anchor 708 is separately formed and comprises a distinct component part from the drive housing 208 (FIG. 7). As illustrated, the routing anchor 708 includes an elongate body 802 having a first or “distal” end 804a and a second or “proximal” end 804b opposite the distal end 804a.

The axial retainment feature 712 may be provided or otherwise defined at the distal end 804a and may be designed to facilitate a tortuous pathway for the wires 702 to traverse. In at least one embodiment, as illustrated, the axial retainment feature 712 may include one or more overhanging bosses 806 designed to prevent vertical movement of the wires 702. In some embodiments, the axial retainment features 712 may be substantially similar in shape and size, and may be defined along a shared centerline. In one or more embodiments, the overhanging portion of each boss 806 may extend laterally in the same direction, but could alternatively extend in opposite lateral directions. The overhanging bosses 806 help to ensure that the wires 702 do not inadvertently escape vertically from the axial retainment feature 712.

In some embodiments, the axial retainment feature 712 may further provide one or more elongate fins 808 (one shown), where each elongate fin 808 interposes axially adjacent overhanging bosses 806. In further embodiments, the overhanging bosses 806 and elongate fins 808 may be aligned perpendicular to a central axis of the routing anchor 708, such that the overhanging bosses 806 are laterally adjacent to the elongate fins 808. The combination of the overhanging bosses 806 and the elongate fin 808 defines a tortuous pathway for the wires 702 to traverse at the distal end 804b of the routing anchor 708. Moreover, the tortuous pathway enables the setting of a desired slack for the wires 702. In particular, the wires 702 may then be set to a desired slack length, following which the wires 702 may be threaded (guided) through a distal-most overhanging boss 806 and around the elongate fin(s) 808, such that the wires 702 are partially maintained in place. The wires 702 may then be threaded around a proximal-most overhanging boss 806, thereby locking the wires 702 in place within the axial retainment feature 712.

In some embodiments, a connector 810 may be provided at the proximal end 804b of the routing anchor 708. The connector 810 may be sized to be operatively coupled to a corresponding receiver (not shown) provided in the slot 710 (FIG. 7) to further constrain the motion of the routing anchor 708 during installation. As such, the connector 810 may enable precise seating of the routing anchor 708 within the drive housing 208, while further securing the routing anchor 708 in place.

In some embodiments, as illustrated, the routing anchor 708 may provide or define a wire channel 812 extending along at least a portion of a length of the body 802 of the routing anchor 708 between the distal and proximal ends 804a,b. The wire channel 812 may be configured to receive and guide the wires 702 therethrough and thereby help constrain vertical motion of the wires 702, such that slack in the wires 702 is prevented from migrating towards moving component parts included within the drive housing 208. As illustrated, the wire channel 812 includes a first or “distal” opening 814a and a second or “proximal” opening 814b, and the wire channel 812 extends between the distal and proximal openings 814a,b. The wires 702 enter/exit the wire channel 812 at the distal and proximal openings 814a,b.

In some embodiments, a plurality of access apertures 816 (three shown) may be defined along a length of the wire channel 812 to provide intermittent access and a viewport into the interior of the wire channel 812. In such embodiments, the access apertures 816 may help threading of the wires 702 through the wire channel 812, without providing sufficient space to enable vertical motion of said wires 702. The access apertures 816 may further enable the formation of the wire channel 812 during injection molding processes, while also providing visual confirmation of the wires 702 and ease of assembly for threading wires 702 therethrough.

In some embodiments, the routing anchor 708 may provide an engagement surface 818 proximally adjacent to the axial retainment feature 712. The engagement surface 818 may taper from the axial retainment feature 712 towards the proximal end 804b at an angle or slope, such that the engagement surface 818 may be received under and engage one or more side walls of the slot 710 (FIG. 7). The engagement surface 818 may enable sliding engagement with the side walls, such that the routing anchor 708 may be distally inserted into the drive housing 208 (FIG. 7) and proximally slid into place. To this end, the engagement surface 818 may provide one or more vertical protrusions, or “bumps”, 820 on a horizontal section of the engagement surface, which may form an interference fit with the one or more side walls of the slot 710 (FIG. 7) to retain the routing anchor 708 in place.

FIG. 9 is another exposed isometric view of the interior of the drive housing 208, according to one or more additional embodiments. Several additional component parts of the drive housing 208 are omitted in FIG. 9 to enable discussion of the remaining component parts. The routing anchor 708 is received within the interior of the slot 710. In the illustrated embodiment, the connector 810 (FIG. 8) provided at the proximal end 804b of the routing anchor 708 is received by and retained beneath a receiver 902 defined within the slot 710. The receiver 902 may comprise, for example, a lateral extension formed within the slot 710. Receiving the connector 810 at the receiver 902 may provide an initial seating of the routing anchor 708 in the drive housing 208, and may help constrain vertical motion of the routing anchor 708 out of the slot 710. Once the connector 810 is received within (beneath) the receiver 902, the routing anchor 708 may then be pressed down into place within the slot 710 and secured with a crush rib fit within the drive housing 208 to fully constrain the routing anchor 708 from any undesired motion. In some embodiments, the engagement surface 818 may further enable the crush rib fit, as the routing anchor 708 is slidingly engaged with the side walls of the slot 710 at a distal end thereof. The crush rib fit can accordingly include the interference fit of the vertical protrusions 820 (FIG. 8) within corresponding slots defined in the slot 710 (not shown).

To further maintain proper routing and constraint of the wires 702 within the drive housing 208, the wire routing system 706 may further include a wire guide 904, separate from but similar in some respects to the axial retainment feature 712. Similar to the axial retainment feature 712, the wire guide 904 may be designed to facilitate a tortuous pathway for the wires 702 to traverse. Unlike the axial retainment feature 712, however, the wire guide 904 may be provided or otherwise defined by the drive housing 208.

More specifically, the wire guide 904 may include a plurality of overhanging bosses 906 designed to prevent vertical movement of the wires 702. The overhanging projection of each overhanging boss 906 may extend in alternating directions, such that the wire guide 904 may include a similar tortuous pathway as compared to the axial retainment feature 712. The overhanging bosses 906 may receive the wires 702 to be tucked under each overhang in a serpentine path, such that movement of the wires 702 is constrained both laterally and vertically.

The wire guide 904 may further include a guide channel entry tab 908 arranged to receive (redirect) the wires 702 upon exiting the routing anchor 708 and the slot 710. The guide channel entry tab 908 may be angled such that the tortuous pathway of the wire guide 904 is extended, and the wires 702 are further constrained between an angled projection of the guide channel entry tab 908 and the vertical boss of the adjacent overhanging boss 906. The guide channel entry tab 908 may be provided on a proximal side of a notch 910 defined in a wall of the slot 710, and through which the wires 702 may enter/exit the slot 710 and the routing anchor 708.

In some embodiments, at least one of the overhanging bosses 906 may define a rear fin 912 that projects laterally away from the overhang thereof. The rear fin 912 may help to secure the electrical assembly 704 (FIG. 7) within the drive housing 208. In particular, the rear fin 912 may provide a bottoming surface for the electrical assembly 704 installed thereabove, such that the rear fin 912 can be used in pressing operations between the wires 702 and the power cable 214 of FIG. 2. The rear fin 912 may provide a platform of increased size, compared to the overhanging bosses 906, such that the bottoming surface for the electrical assembly 704 includes an increased area. The increased surface area provided by the rear fin 912 may further increase the strength of the bottoming surface to enable pressing operations to be performed when connecting the wires 702 to the electrical assembly 704.

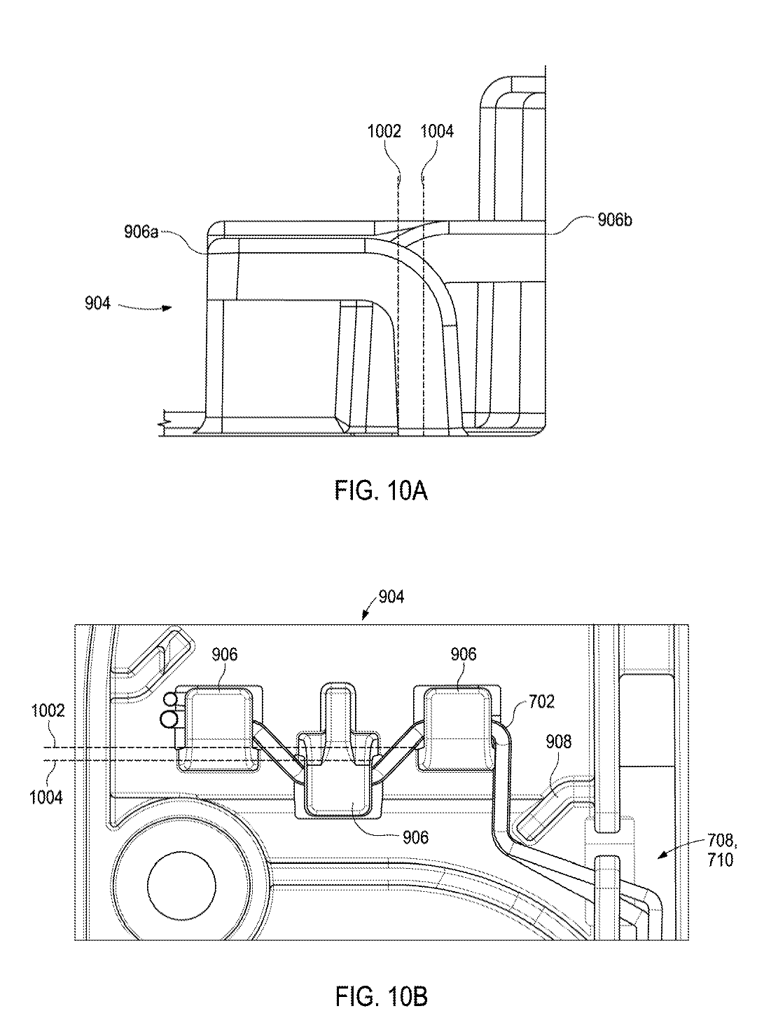

FIG. 10A is an enlarged side view of a portion of the wire guide 904, according to one or more embodiments. In the illustrated embodiment, first and second overhanging bosses are depicted as 906a and 906b, and are shown to laterally overlap to help form the tortuous pathway of the wire guide 904. The first overhanging boss 906a includes a first vertical interior surface 1002 (graphically depicted as a projected, dashed line) against which the wires 702 may be held and retained. Similarly, the second overhanging boss 906b includes a second vertical interior surface 1004 (graphically depicted as a projected, dashed line) for retaining the wires 702. In the illustrated embodiment, the space (gap) between the first and second vertical interior surfaces 1002, 1004 provides an overlapping thickness between the first and second overhanging bosses 906a,b. As the wires 702 are threaded (guided) through the first and second overhanging bosses 906a,b, motion of the wires 702 may be constrained by said overlap between the first and second vertical interior surfaces 1002, 1004, such that there is no straight path therebetween. In some embodiments, however, the first and second vertical interior surface 1002, 1004 may be aligned along a centerline, or may be laterally offset to define a straight path therebetween, without departing from the scope of the present disclosure.

FIG. 10B is an enlarged top view of the wire guide 904 depicting the overhanging bosses 906 with the wires 702 constrained therein, according to one or more embodiments. As illustrated, the wires 702 are required to traverse a tortuous pathway as guided (threaded) through the overhanging bosses 906, and being re-oriented between the alternating first and second vertical interior surfaces 1002, 1004. The tortuous path of the wire guide 904 can accordingly limit travel of the wires 702 through the overhangs of the overhanging bosses 906, while further limiting any lateral translation of the wires 702 through the multiple (e.g., three) alternating, laterally offset, vertical interior surfaces 1002, 1004 of the overhanging bosses 906.

To complete the threading of the wires 702 through the tortuous path of the wire guide 904, the wires 702 may continue towards the guide channel entry tab 908 to enter into the slot 710 and the routing anchor 708 arranged therein. The guide channel entry tab 908 may accordingly provide a fourth surface against which the wires 702 may be maintained in tension. As shown in the illustrated embodiment, the wires 702 may be routed around and pivot against the laterally adjacent overhanging boss 906 as the wires 702 are directed towards the guide channel entry tab 908, further defining the serpentine path of the tortuous path for the wire guide 904.

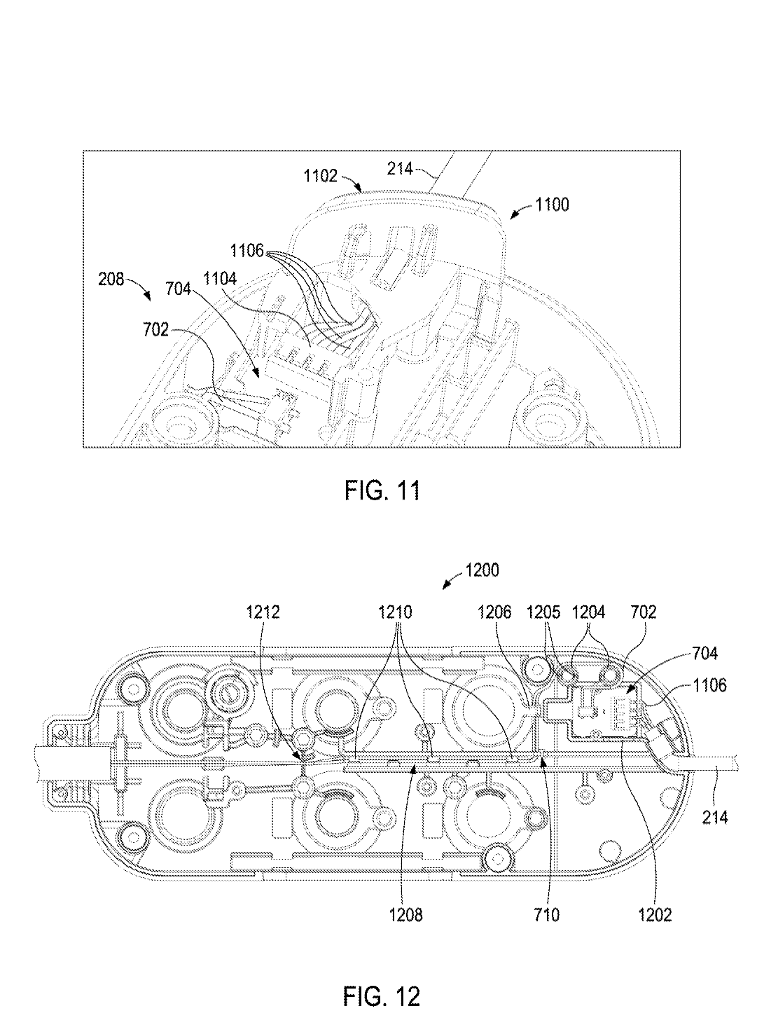

FIG. 11 is a partial isometric view of an example electrical connector 1100 installed within the drive housing 208, according to one or more embodiments. The electrical connector 1100 may provide an anchoring and endpoint for the wires 702, while also providing an interface between the power cable 214 and the wires 702 to complete an electrical circuit with the generator 216 (FIG. 2). The electrical connector 1100 may be received within a slot provided at the proximal end of the drive housing 208, and, in some embodiments, may include a clamshell assembly 1102.

The electrical connector 1100 may include the electrical assembly 704, which may be secured within the clamshell assembly 1102. The electrical assembly 704 may comprise, for example, a printed circuit board (PCB) including an electrical junction 1104 that provides a connection point between the electrical assembly 704 and one or more generator cables 1106 provided through the power cable 214. The electrical junction 1104 may retain the ends of each generator cable 1106 therein to provide power and/or other signals to the electrical assembly 704. Positioning the clamshell assembly 1102 within the drive housing 208, as well as the location of the electrical junction 1104, may further provide strain relief for the power cable 214 and the generator cables 1106. In some embodiments, the clamshell assembly 1102 may include a crimped metal component to provide the strain relief for the power cable 214. The crimped metal component may be engaged within the clamshell assembly 1102, which itself is captured within the drive housing 208, to thus distributed any applied loads throughout these interconnected components.

As shown in the illustrated embodiment, the wires 702 extend from the electrical assembly 704 and may be threaded down below towards the drive housing 208 and the wire guide 904 of FIGS. 9-10B. As previously discussed, the overhanging bosses 906 (FIGS. 9-10B) and the rear fin 912 (FIGS. 9-10B) may provide a bottoming surface for the electrical assembly 704. In some embodiments, the electrical assembly 704 may rest atop these components to provide further points of contact within the drive housing 208. This further support from below the clamshell assembly 1102 may enable the application of downward forces during potential press operations, thus preventing motion or slippage of the clamshell assembly 1102 through the further support surfaces.

The combined properties of the routing anchor 708 (FIGS. 7-9), the wire guide 904 (FIGS. 9-10), and the electrical connector 1100 of FIG. 11 may provide a routing pathway that maintains the wires 702 in a predictable position to prevent binding or damage during the operation of the surgical tool 200 of FIG. 2. The reduced strain on the power cable 214, the first and second tortuous pathways, and the wire channel 812 (FIGS. 8 and 9) can prevent wear of the various electrical connectors, while also safely enabling the setting of a desired slack for the articulation of any connected tooling.

FIG. 12 is a top view of the interior of another example drive housing 1200, according to one or more additional embodiments. The drive housing 1200 may be similar in some respects to the drive housing 208 and, therefore, may be best understood with reference thereto, where like numerals will correspond to like components not described again in detail. Similar to the drive housing 208, for example, the drive housing 1200 may receive the power cable 214, and may receive the generator cables 1106 on the electrical assembly 704 to provide power and/or signals to the wires 702.

Unlike the drive housing 208, however, the drive housing 1200 may include a raised or embossed border 1202 for receiving and locating the electrical assembly 704 within the drive housing 1200 and providing attachment points for fastening the electrical assembly 704 therein. Moreover, the wires 702 may extend from the electrical assembly 704 and towards one or more cylindrical bosses 1204 defined by the drive housing 1200. The cylindrical bosses 1204 may provide rounded external surfaces about which the wires 702 may be routed within the drive housing 1200. The rounded shape of the cylindrical bosses 1204 may help set a desired slack of the wires 702, such that the wires 702 may be wrapped around the cylindrical bosses 1204 as needed to take up any excess slack. In some embodiments, the top surface of each cylindrical bosses 1204 may define one or more notches 1205 therein, such that the wires 702 may be received therein with an interference fit and to maintain positioning of the wires 702 after wrapping. From the cylindrical bosses 1204, the wires 702 may extend towards the slot 710.

The drive housing 1200 may further include a protruding overhang 1206 that provides a hooked protrusion for receiving and holding the wires 702. The hooked protrusion may include an upper surface that prevents vertical movement of the wires 702, and a downward projection from the upper surface to maintain the wires 702 between the downward projection and the drive housing 1200 itself. In some embodiments, the protruding overhang 1206 may be molded as part of the drive housing 1200 at or near the location of one of the drive inputs 506a-f (FIG. 5). The combined path of the cylindrical bosses 1204 and the protruding overhang 1206 may provide a similar tortuous pathway to the embodiments of FIGS. 7-10, such that the wires 702 may be similarly retained and protected via the drive housing 1200.

The drive housing 1200 omits the routing anchor 708 (FIG. 7), but the slot 710 may operate as a type of routing anchor and provide or otherwise define a wire channel 1208 extending along at least a portion of the length of the slot 710. The wire channel 1208 may be configured to receive the wires 702 therethrough and help constrain vertical movement of the wires 702, such that slack in the wires 702 is prevented from extending vertically towards the moving component parts housed within the drive housing 1200. To accomplish this, the wire channel 1208 includes a plurality of tabs 1210 extending laterally into the interior of the slot 710, and under which the wires 702 can be routed and maintained. The tabs 1210 may be spaced and shaped such that the wires 702 may be prevented from moving vertically out of the slot 710 and towards any moving components. Accordingly, the drive housing 1200 may be characterized as including an integrally formed routing anchor and corresponding wire channel 1208 using the tabs 1210 for maintaining the wires 702 within the slot 710.

The drive housing 1200 may further provide an additional axial retainment feature 1212 located at or near a distal end of the slot 710. The axial retainment feature 1212 may define a vertically tapering slit that tapers towards a bottom edge thereof. The tapering slit may be sized to receive the wires 702 at a top end, and the wires 702 may then be lowered into the tapering slit until tightly secured within the axial retainment feature 1212. The wires 702 may then continue through the drive housing 1200 and towards any connected end effectors or tooling.

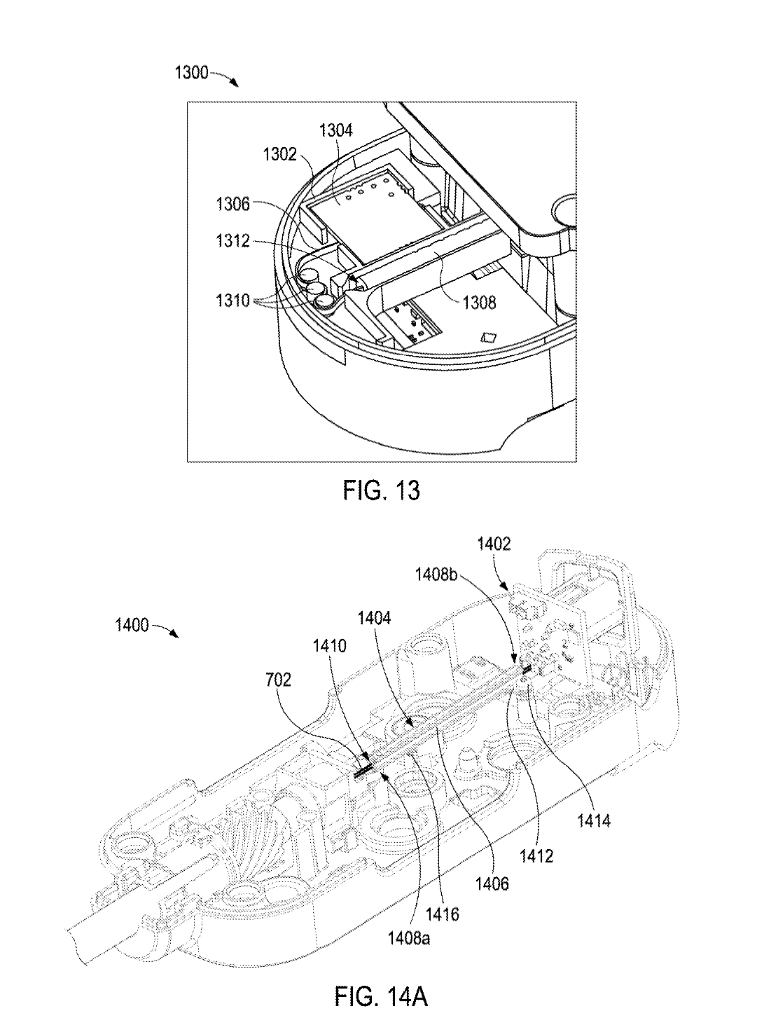

FIG. 13 is a partial isometric view of another example drive housing 1300, in accordance with one or more additional embodiments. The drive housing 1300 may be similar in some respects to one or both of the drive housings 208, 1200 and, therefore, may be best understood with reference thereto. Similar to the drive housing 1200, for example, the drive housing 1300 includes an embossed border 1302 for locating and maintaining the position of an electrical assembly 1304, similar to the electrical assembly 704 (FIG. 12).

The drive housing 1300 includes one or more wires 1306 (one shown) extending from the electrical assembly 704 and towards a driven gear 1308 within the drive housing 1300. The wire 1306 may be similar to the wires 702 of FIGS. 7-9 in that the wire 1306 is able to convey electrical power from the electrical assembly 1304 to the end effector 204 (FIGS. 2 and 4), for example. The wire 1306 in FIG. 13, however, comprises a flex circuit configured to extend down a center portion of the driven gear 1308. The driven gear 1308 may be the same or similar to the driven gear 606b of FIGS. 6 and 7 and, therefore, may comprise a linearly actuating member referred to herein as a “knife rack” or “firing rack”.

As illustrated, the drive housing 1300 includes or otherwise defines axial retainment feature 1310 that provides a tortuous pathway through which the wire 1306 may traverse. In the illustrated embodiment, the axial retainment feature 1310 comprises a plurality of cylindrical bosses arranged in series. The cylindrical bosses may enable multiple wraps of the wire 1306 thereabout such that slack in the wire 1306 may be controlled and adjusted within the drive housing 1300.

In the illustrated embodiment, the wire 1306 may following a serpentine path through the axial retainment feature 1310, and the wire 1306 may extend from the axial retainment feature 1310 towards the driven gear 1308. The driven gear 1308 may define a wire channel 1312 sized to receive and guide the wire 1306. The wire channel 1312 provides a safe pathway for the wire 1306 to travel without interference from the driven gear 1308, as the driven gear 1308 may translate parallel to a direction of the wire 1306. As such, the combination of the axial retainment feature 1310 and the wire channel 1312 may provide a routing pathway that protects the wire 1306 from damage while maintaining a predictable position of the wire 1306 within the alternate drive housing 1300. Accordingly, the driven gear 1308 moves independent of the wire 1306, such the driven gear 1308 translates (reciprocates) without interference or subsequent translation of the wire 1306.

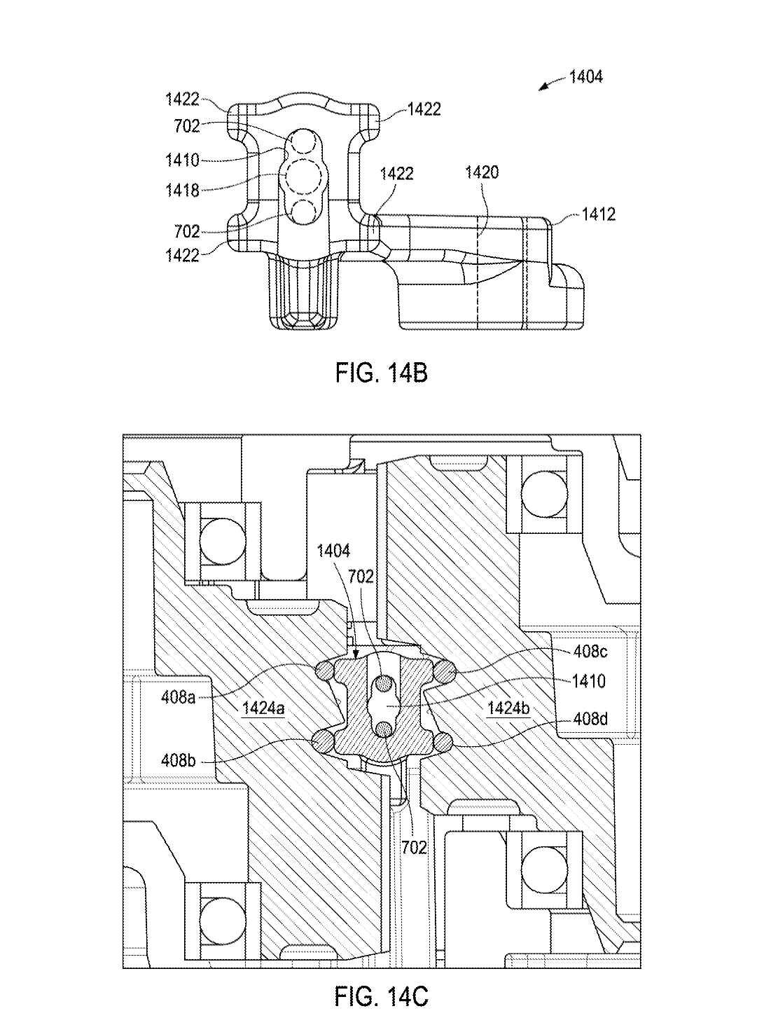

FIG. 14A is an isometric view of another example drive housing 1400, in accordance with one or more additional embodiments. The drive housing 1400 may be similar in some respects to any of the drive housings 208, 1200, 1300 described herein and, therefore, may be best understood with reference thereto. Similar to the drive housings 208, 1200, for example, the drive housing 1400 includes an electrical assembly 1402 and the one or more wires 702 extend within the drive housing 1400 and terminate at the electrical assembly 704. Moreover, similar to the drive housing 208, the drive housing 1400 further includes a routing anchor 1404 operatively coupled to the drive housing 1400 and configured to receive and protect the wires 702 from contacting various moving component parts included within the drive housing 1400.

As illustrated, the routing anchor 1404 provides an elongate body 1406 having opposing distal and proximal ends 1408a and 1408b, and a wire channel 1410 is defined within the body 1406 and extends between the distal and proximal ends 1408a,b. The wire channel 1410 is sized to receive and guide the wires 702 to/from the electrical assembly 704 within the drive housing 1400.

In some embodiments, the routing anchor 1404 includes a mounting bracket 1412 extending laterally from the body 1406 at or near the proximal end 1408b. The mounting bracket 1412 may define a central aperture sized to receive a fastener 1414 used to removably attach the routing anchor 1404 to the drive housing 1400.

In at least one embodiment, the distal end 1408a of the routing anchor 1404 may also be operatively coupled to the drive housing 1400, thereby constraining the routing anchor 1404 at each end 1408a,b. More particularly, a portion of the routing anchor 1404 may extend from the bottom of the body 1406 to be received at a vertical column or pillar 1416 provided by the drive housing 1400. The vertical pillar 1416 includes a slot or groove sized to receive the portion of the routing anchor 1404. In one or more embodiments, the portion of the drive housing 1400 may be received at the vertical pillar 1416 via a press fit tab or interference fit to thereby prevent motion thereof.

FIG. 14B is an enlarged end view of the routing anchor 1404, according to one or more embodiments. As illustrated, the routing anchor 1404 defines the wire channel 1410 sized to receive and guide the wires 702 (shown in dashed lines). In monopolar embodiments, the wires 702 may be replaced with a single wire 1418 (shown in dashed lines). As illustrated, the wire channel 1410 may include geometry configured to receive and seat the wires 702 or the single wire 1418. More specifically, the wire channel 1410 may define arcuate or curved surfaces that approximate the diameter of the wires 702 and/or the single wire 1418. The routing anchor 1404 further includes the mounting bracket 1412, which may define a central aperture 1420 sized to receive the fastener 1414 (FIG. 14A) used to removably attach the routing anchor 1404 to the drive housing 1400 (FIG. 14A).

In some embodiments, the routing anchor 1404 may further include one or more lateral projections 1422 (four shown) extending laterally from the body 1406 of the routing anchor 1404. Each lateral projection 1422 may be configured to align with and help prevent derailment of corresponding drive cables (e.g., the drive cables 408a-d of FIG. 4). In the illustrated embodiment, two lateral projections 1422 are provided on each side of the routing anchor 1404 and are vertically offset from each other.

FIG. 14C is an enlarged cross-sectional view of a portion of the drive housing 1400, according to one or more embodiments. As illustrated, the routing anchor 1404 is arranged between opposing structural components 1424a and 1424b, and the wires 702 are received within the wire channel 1410 and are thereby protected from moving components included within the drive housing 1400. For example, one or both of the structural components 1424a,b may comprise a moving component, such as a pulley or a gear.

In the illustrated embodiment, each lateral projection 1422 is arranged to align with a corresponding drive cable 408a-d. In operation, the lateral projections 1422 help to maintain the drive cables 408a-d extending in a straight path, and thereby prevent derailment of the drive cables 408a-d.

Embodiments disclosed herein include:

A. A surgical tool including a drive housing including an electrical assembly housed therein, an elongate shaft extending from the drive housing, an end effector arranged at a distal end of the elongate shaft, one or more wires extending from the end effector to the drive housing and terminating at the electrical assembly, and a wire routing system provided within the drive housing. The wire routing system includes an axial retainment feature including one or more overhanging bosses that define a tortuous pathway for the one or more wires to traverse, and a wire channel that guides the one or more wires toward the electrical assembly.

B. A method of securing one or more wires of a surgical tool including receiving the one or more wires within a drive housing of the surgical tool. The surgical includes an electrical assembly housed within the drive housing, an elongate shaft extending from the drive housing, and an end effector arranged at a distal end of the elongate shaft, the one or more wires extending from the end effector to the drive housing. The method further includes routing the one or more wires through a wire routing system provided within the drive housing. The wire routing system includes an axial retainment feature including one or more overhanging bosses that define a tortuous pathway for the one or more wires to traverse, and a wire channel that guides the one or more wires toward the electrical assembly. The method further includes receiving the one or more wires at the electrical assembly.

Each of embodiments A and B may have one or more of the following additional elements in any combination: Element 1: wherein the wire routing system includes a routing anchor arranged within the drive housing and including an elongate body having opposing first and second ends, wherein the axial retainment feature is provided at the first end, and wherein the wire channel is defined by the routing anchor and extending along at least a portion of the body between the first and second ends. Element 2: wherein the drive housing defines a slot and the routing anchor is at least partially arranged within the slot. Element 3: wherein the routing anchor further includes a connector provided at the second end and configured to be operatively coupled to a receiver provided in the slot. Element 4: wherein the routing anchor further includes an engagement surface at or near the axial retainment feature and receivable under one or more side walls of the slot to provide an interference fit between the routing anchor the drive housing. Element 5: wherein the one or more overhanging bosses comprise first and second overhanging bosses, and the axial retainment feature further includes a lateral fin interposing the first and second overhanging bosses. Element 6: wherein the one or more overhanging bosses comprise one or more first overhanging bosses, and the tortuous pathway comprises a first tortuous pathway, the surgical tool further including a wire guide defined by the drive housing including one or more second overhanging bosses that define a second tortuous pathway for the one or more wires to traverse, wherein the axial retainment feature feeds the one or more wires into the wire channel, and wherein the wire channel feeds the one or more wires to the wire guide. Element 7: wherein the drive housing defines a slot through which the wire channel extends, and the wire guide further includes a guide channel entry tab extending from the slot and arranged to redirect the one or more wires upon exiting the wire channel. Element 8: wherein at least one of the one or more second overhanging bosses defines a rear fin engageable with the electrical assembly. Element 9: wherein the wire routing system further includes one or more cylindrical bosses defined by the drive housing and about which the one or more wires are routed.

Element 10: wherein the drive housing defines a slot and the slot comprises the wire channel, and wherein a plurality of tabs extend laterally into the slot and the one or more wires are routed beneath the plurality of tabs. Element 11: the surgical tool further including a wire guide located at a distal end of the slot, the wire guide defining a vertically tapering slit sized to receive and secure the one or more wires. Element 12: wherein the wire routing system includes a routing anchor arranged within the drive housing and including an elongate body having opposing first and second ends, wherein the axial retainment feature is provided at the first end, and wherein the wire channel is defined by the routing anchor and extending along at least a portion of the body between the first and second ends, and wherein routing the one or more wires through the wire routing system comprises routing the one or more wires through the tortuous pathway of the one or more overhanging bosses, receiving the one or more wires at the wire channel from the one or more overhanging bosses, and routing the one or more wires through the wire channel. Element 13: wherein the drive housing defines a slot and routing the one or more wires through the wire routing system is preceded by receiving the routing anchor is at least partially within the slot. Element 14: wherein the one or more overhanging bosses comprise first and second overhanging bosses, and the axial retainment feature further includes a lateral fin interposing the first and second overhanging bosses. Element 15: wherein the one or more overhanging bosses comprise one or more first overhanging bosses and the tortuous pathway comprises a first tortuous pathway, the method further comprising feeding the one or more wires into the wire channel from the axial retainment feature, and feeding the one or more wires from the wire channel to a wire guide defined by the drive housing, the wire guide including one or more second overhanging bosses that define a second tortuous pathway for the one or more wires to traverse. Element 16: wherein the drive housing defines a slot through which the wire channel extends, and the wire guide further includes a guide channel entry tab extending from the slot, the method further comprising redirecting the one or more wires upon exiting the wire channel with the guide channel entry tab. Element 17: wherein at least one of the one or more second overhanging bosses defines a rear fin, the method further comprising engaging the electrical assembly on the rear fin. Element 18: wherein the wire routing system further includes one or more cylindrical bosses defined by the drive housing, the method further comprising routing the one or more wires about the one or more cylindrical bosses.

By way of non-limiting example, exemplary combinations applicable to A and B include: Element 1 with Element 2; Element 2 with Element 3; Element 3 with Element 4; Element 6 with Element 7; Element 6 with Element 8; Element 10 with Element 11; Element 12 with Element 13; Element 12 with Element 14; Element 15 with Element 16; and Element 15 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A surgical tool, comprising:
a drive housing including an electrical assembly housed therein;
an elongate shaft extending from the drive housing;
an end effector arranged at a distal end of the elongate shaft;
one or more wires extending from the end effector to the drive housing and terminating at the electrical assembly; and
a wire routing system provided within the drive housing and including:
an axial retainment feature including one or more overhanging bosses that define a tortuous pathway for the one or more wires to traverse; and
a wire channel that guides the one or more wires toward the electrical assembly.

2. The surgical tool of claim 1, wherein the wire routing system includes a routing anchor arranged within the drive housing and including an elongate body having opposing first and second ends, wherein the axial retainment feature is provided at the first end, and wherein the wire channel is defined by the routing anchor and extending along at least a portion of the body between the first and second ends.

3. The surgical tool of claim 2, wherein the drive housing defines a slot and the routing anchor is at least partially arranged within the slot.

4. The surgical tool of claim 3, wherein the routing anchor further includes a connector provided at the second end and configured to be operatively coupled to a receiver provided in the slot.

5. The surgical tool of claim 4, wherein the routing anchor further includes an engagement surface at or near the axial retainment feature and receivable under one or more side walls of the slot to provide an interference fit between the routing anchor and the drive housing.

6. The surgical tool of claim 1, wherein the one or more overhanging bosses comprise first and second overhanging bosses, and the axial retainment feature further includes a lateral fin interposing the first and second overhanging bosses.

7. The surgical tool of claim 1, wherein the one or more overhanging bosses comprise one or more first overhanging bosses, and the tortuous pathway comprises a first tortuous pathway, the surgical tool further including:
a wire guide defined by the drive housing including one or more second overhanging bosses that define a second tortuous pathway for the one or more wires to traverse,
wherein the axial retainment feature feeds the one or more wires into the wire channel, and
wherein the wire channel feeds the one or more wires to the wire guide.

8. The surgical tool of claim 7, wherein the drive housing defines a slot through which the wire channel extends, and the wire guide further includes a guide channel entry tab extending from the slot and arranged to redirect the one or more wires upon exiting the wire channel.

9. The surgical tool of claim 7, wherein at least one of the one or more second overhanging bosses defines a rear fin engageable with the electrical assembly.

10. The surgical tool of claim 1, wherein the wire routing system further includes one or more cylindrical bosses defined by the drive housing and about which the one or more wires are routed.

11. The surgical tool of claim 1, wherein the drive housing defines a slot and the slot comprises the wire channel, and wherein a plurality of tabs extend laterally into the slot and the one or more wires are routed beneath the plurality of tabs.

12. The surgical tool of claim 11, the surgical tool further including a wire guide located at a distal end of the slot, the wire guide defining a vertically tapering slit sized to receive and secure the one or more wires.

13. A method of securing one or more wires of a surgical tool, comprising:
receiving the one or more wires within a drive housing of the surgical tool, the surgical tool including:
an electrical assembly housed within the drive housing;
an elongate shaft extending from the drive housing; and
an end effector arranged at a distal end of the elongate shaft, the one or more wires extending from the end effector to the drive housing;
routing the one or more wires through a wire routing system provided within the drive housing, the wire routing system including:
an axial retainment feature including one or more overhanging bosses that define a tortuous pathway for the one or more wires to traverse; and
a wire channel that guides the one or more wires toward the electrical assembly; and
receiving the one or more wires at the electrical assembly.

14. The method of claim 13, wherein the wire routing system includes a routing anchor arranged within the drive housing and including an elongate body having opposing first and second ends, wherein the axial retainment feature is provided at the first end, and wherein the wire channel is defined by the routing anchor and extending along at least a portion of the body between the first and second ends, and wherein routing the one or more wires through the wire routing system comprises:
routing the one or more wires through the tortuous pathway of the one or more overhanging bosses;
receiving the one or more wires at the wire channel from the one or more overhanging bosses; and
routing the one or more wires through the wire channel.

15. The method of claim 14, wherein the drive housing defines a slot and routing the one or more wires through the wire routing system is preceded by receiving the routing anchor is at least partially within the slot.

16. The method of claim 14, wherein the one or more overhanging bosses comprise first and second overhanging bosses, and the axial retainment feature further includes a lateral fin interposing the first and second overhanging bosses.

17. The method of claim 13, wherein the one or more overhanging bosses comprise one or more first overhanging bosses and the tortuous pathway comprises a first tortuous pathway, the method further comprising:
feeding the one or more wires into the wire channel from the axial retainment feature; and
feeding the one or more wires from the wire channel to a wire guide defined by the drive housing, the wire guide including one or more second overhanging bosses that define a second tortuous pathway for the one or more wires to traverse.

18. The method of claim 17, wherein the drive housing defines a slot through which the wire channel extends, and the wire guide further includes a guide channel entry tab extending from the slot, the method further comprising redirecting the one or more wires upon exiting the wire channel with the guide channel entry tab.

19. The method of claim 17, wherein at least one of the one or more second overhanging bosses defines a rear fin, the method further comprising engaging the electrical assembly on the rear fin.

20. The method of claim 13, wherein the wire routing system further includes one or more cylindrical bosses defined by the drive housing, the method further comprising routing the one or more wires about the one or more cylindrical bosses.
